(19) United States
(12) Patent Application Publication
(10) Pub. No.: US 2007/0066767 A1
(43) Pub. Date: Mar. 22, 2007

(54) Polyalkylene glycols

(75) Inventors: Naohiko Itayama, Takatsuki-shi (JP); Hiroshi Yamamoto, Kobe-shi (JP); Tsutomu Yuasa, Osaka-shi (JP)

Correspondence Address: CONNOLLY BOVE LODGE & HUTZ LLP, P.O. BOX 2207, WILMINGTON, DE, 19899-2207, US

(73) Assignee: Nippon Shokubai Co., Ltd., Osaka-shi (JP)

(21) Appl. No.: 11/513,229

(22) Filed: Aug. 31, 2006

(30) Foreign Application Priority Data
Aug. 31, 2005 (JP) 2005-252877

(51) Int. Cl. C08F 283/04; C07C 43/11

(52) U.S. Cl. 525/453; 568/679

(57) ABSTRACT

The present invention provides polyalkylene glycols hardly containing impurities and a production method thereof. Specifically, the present invention provides: polyalkylene glycols hardly containing diols such as polyethylene glycol, diesters, halogens, metals, water, coloring-causing substances; and a production method thereof. A polymerizable polyalkylene glycol satisfying the characteristics (1) and/or (2): (1) a polymerizable dimer content is 0.001 to 10% by weight and (2): a metal content is 50 ppm or less. It is more preferable that the polymerizable dimer content is 0.001 to 3.5% by weight, and it is still more preferable that the above-mentioned characteristics (1) and (2) are satisfied.

### TECHNICAL FIELD

The present invention relates to polyalkylene glycols and production method thereof. More specifically, the present invention relates to: polyalkylene glycols useful as a production raw material that can be used in various applications, for example, various polymer materials, concrete admixtures, cohesive agents, adhesive agents, coating materials, cosmetics additives, and various dispersants such as inorganic dispersants; and a production method thereof.

### BACKGROUND ART

Polyalkylene glycols are compounds that can be used as various industrial chemical raw materials. Particularly polyalkylene glycols having an unsaturated bond are useful as monomers forming polymers used in various applications. As a production method of such polyalkylene glycols, reaction of synthesizing a polymerizable polyalkylene glycol by directly adding an alkylene oxide (AO) to hydroxyalkyl (meth)acrylate as an initiator has been known, for example. In such reaction, a catalyst for adding the alkylene oxide is used and the alkylene oxide is reacted with active hydrogen of the initiator, or with a terminal hydroxyl group of the bydroxyalkyl (meth)acrylate, and thereby the alkylene oxide is further added. Thereby, a compound having a polyalkylene glycol chain and a terminal unsaturated group derived from the hydroxyalkyl (meth)acrylate is generated.

As a conventional production method of a (meth)acrylate monomer having a polyalkylene glycol chain, for example, Japanese Kohyo Publication No. 2001-514280 (pages 2 to 7) discloses that at least one carbon-carbon unsaturated part and an initiator molecule having: at least one functional group capable of being oxyalkylated by an alkylene oxide; and one or less tree carboxylic acid group are oxyalkylated by an alkylene oxide in the presence of a double metallocyanide complex catalyst, and thereby, hydroxyl-, and unsaturated-functional polyoxyalkylene polyether is produced. Japanese Kohyo Publication No. 2003-504468 (pages 2 and 3) discloses a method for producing polyether from a mixture of an initiator compound, ethylene oxide and a metallocyanide catalyst complex.

Japanese Kokai Publication No. 2005-170814 (page 1) discloses a method for producing polyethylene glycol methacrylate, in which a boron trifluoride compound is used as a catalyst, and ring-opening polymerization of ethylene oxide with 2-hydroxyethyl methacrylate is performed, and then the boron trifluoride compound is treated with an adsorbent. However, metal cyano complex compounds and tin chlorides including so-called DMC catalyst, and Lewis acid catalysts including boron trifluoride compounds are dissolved in raw materials and used. Therefore, in order to remove impurities derived from such catalysts, extraction with water and organic solvents or removal with adsorbents needs to be performed after the reaction. Therefore, products with high purity are difficult to produce in economic terms.

In addition, Japanese Kokoku Publication No. Hei-07-10801 (page 1) discloses a method for producing (meth)acrylate by addition reaction of an alkylene oxide in the presence of activated clay, for example. Also, Japanese Kokoku Publication No. Sho-52-30469 (page 1) discloses a method for producing polyalkylene glycol monoacrylate or monomethacrylate by addition of an alkylene oxide in the presence of a catalytic amount of tin tetrachloride.

However, the activated clay has low catalyst activity and the reaction requires much time. And use of SnCl₄ produces many diesters as a by product generated by esterification of both ends of a generated product. Such diesters act as a cross-linking agent because such diesters contain an unsaturated group in the structures at both terminals. Therefore, such (meth)acrylate monomers having a polyalkylene glycol chain containing many diesters as an impurity fail to effectively exhibit the functions in various applications. Therefore, a production method capable of: effectively adding an alkylene oxide to an initiator; and suppressing a content of adverse components such as diesters in produced polyalkylena glycols, has been desired in the production of polyalkylene glycols.

### SUMMARY OF THE INVENTION

The present invention provides polyalkylene glycols hardly containing impurities and a production method thereof. More specifically, the present invention relates to: polyalkylene glycols hardly containing diols such as polyethylene glycol, diesters, halogens, metals, water, and coloring-causing substances; and a production method thereof. Polyalkylene glycol mono(meth)acrylates are generally used as a raw material for (meth)acrylate resin and photo-curable resins in order to improve the adhesion and provide compatibility and flexibility for such resins. Particularly, polyalkylene glycol mono(meth)acrylates having a small molar number of addition of alkylene oxides are used as a reactive diluent. Such polyalkylene glycol mono(meth)acrylates having a small molar number of addition of alkylene oxides can provide materials with high storage modulus at room temperature because of the characteristics such as low viscosity. However, it has been difficult to remove diols such as polyethylene glycol, diesters, halogens, metals, water, coloring-causing substances in conventional techniques, which fails to produce polyalkylene glycol mono(meth)acrylates with high quality. Particularly in urethane (meth)acrylate used in electron materials and the like, it has been impossible to prevent inhibition reaction, increase in viscosity, gelling, and reduction in dynamic viscoelasticity, which are caused by these impurities. Such circumstances have been problems.

The present inventors have made various investigations on production methods of polyalkylene glycols capable of having an unsaturated group in the presence of a catalyst. The inventors noted that a method for producing a polyalkylene glycol by reacting an alkylene oxide with an unsaturated initiator in the presence of a catalyst is industrially useful. The inventors conducted catalyst screening for developing a catalyst suitable for such a production method and found that solid acid catalysts are effective. Therefore, the above-mentioned problems have been admirably solved. The inventors found that use of solid acids as the catalyst is particularly effective in reaction of synthesizing a polymerizable polyethylene glycol (PEG) by directly adding an alkylene oxide (particularly ethylene oxide) to an unsaturated initiator such as hydroxyalkyl (meth)acrylate, and that if, among the solid acids, crystalline metal oxides, particularly zeolites, or aluminum compounds having a 5-coordination structure of aluminum are used as the catalyst, more advantageous effects can be exhibited.

The present inventors found that the above-mentioned solid catalyst is used to perform addition of an alkylene oxide and then the catalyst is separated by a conventional method, and thereby a polyalkylene glycol mono(meth)acrylate with high purity is produced. Also, the inventors found that the above-mentioned solid catalyst is used to perform addition of an alkylene oxide and then the catalyst is separated by a conventional method, and then, from residues obtained by a conventional method such as distillation, a polyalkylene glycol mono(meth)acrylate with high purity can be produced. Thereby, the present invention has been completed.

That is, the present invention relates to a polymerizable polyalkylene glycol satisfying the following characteristics (1) and/or (2):

(1) a polymerizable dimer content is 0.001 to 10% by weight; and

(2) a metal content is 50 ppm or less.

The present invention also relates to a method for producing a polyalkylene glycol by reacting an alkylene oxide with an unsaturated initiator, wherein at least one solid acid catalyst selected from the group consisting of crystalline metal oxides and aluminum compounds having a 5-coordination structure of aluminum.

### BRIEF DESCRIPTION OF THE DRAWINGS

[FIG. 1]

FIG. 1 is a view showing a molecular weight distribution of a polymer produced in Example 3.

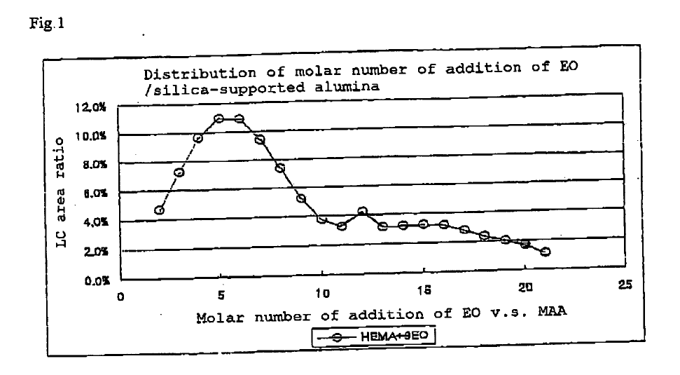

### DETAILED DESCRIPTION OF THE INVENTION

The present invention is described in more detail below.

The terms “or more” and “or less” in the present description mean that the relevant value is included. That is, the term “or more” means that the relevant value and the relevant value or more are included. In the present invention, the above-mentioned production method is firstly mentioned and then the polymerizable polyalkylene glycol is mentioned.

The terms “or more” and “or less” in the present description means that the relevant value is included. That is, the term “or more” means that the relevant value and the relevant value or more are included.

It is preferable in the above-mentioned production method of polyalkylene glycols that a solid acid catalyst is used as a catalyst and an alkylene oxide is reacted with an unsaturated initiator. Use of the solid acid makes it possible to directly add an alkylene oxide to an unsaturated initiator, and thereby a polymerizable polyalkylene glycol can be produced.

The preferable embodiments of the present invention include an embodiment in which the above-mentioned solid acid catalyst is a crystalline metal oxide dispersing in a reaction solution and existing as a solid. Preferable examples of such a solid acid catalyst in the present invention include silica, alumina, silica alumina, various zeolites, activated carbon, kieselguhr, zirconium oxide, rutile titanium dioxide, tin oxide, lead oxide, sulfur oxides consisting of metals belonging to 6 to 13 Groups, composite oxides containing at least one element elected from the group consisting of elements belonging to 3 to 6 and 12 to 15 Groups, composite oxides consisting of silicon and at least one metal, crystalline metalosilicate, zirconia, titania, and magnesia. The solid acid catalyst is not limited thereto.

The above-mentioned crystalline metal oxide is not specially limited as long as it can be preferably used in the production method of the present invention. In addition to the above-mentioned compounds, silica, alumina, crystalline metalosilicate, and zeolites may be specifically mentioned. Among them, zeolites are preferable. β- and γ-zeolites are preferable as the zeolites, and β-zeolite is more preferable. Zeolites have advantages of showing high activity in alkylene oxide (particularly ethylene oxide) addition reaction and hardly generating polymerizable dimers such as diesters as a byproduct.

The above-mentioned crystalline metal oxide has an acid amount of 0.05 mmol NH₃/g or more, and more preferably 0.1 mmol NH₃/g or more, and still more preferably 0.3 mmol NH₃/g or more, and particularly preferably 0.5 mmol NH₃/g or more. These acid amounts are preferably measured by ammonia TPD method.

The preferable embodiments of the present invention include an embodiment in which, in the above-mentioned production method of polyalkylene glycols, an aluminum compound having a 5-coordination structure of aluminum is used as the above-mentioned solid acid catalyst and an alkylene oxide is reacted with an unsaturated initiator. Thereby, the alkylene oxide is directly added to the unsaturated initiator to produce a polymerizable polyalkylene glycol effectively.

It is preferable in the above-mentioned aluminum compound that the peak area at 25 to 35 ppm in 27Al-NMR chart accounts for 40% or more relative to a total of the peak areas at 0 to 10, 25 to 35 and 45 to 55 ppm. Such an aluminum compound has high activity and therefore can effectively produce a polyalkylene glycol in the production method of polyalkylene glycols. This is because the aluminum compound contains a certain or more aluminum having a 5-coordination structure. Generally, aluminum (III) is known to have a coordination number of 6 because the electron orbit involved in the coordination bond is d²sp³ (octahedron bond). In contrast, γ-alumina is known to partly have a 4-coordination structure in addition to 6-coordination structure in which all coordination-capable electron orbits are occupied. If aluminum in such γ-alumina is substituted with another element, so-called coordination unsaturation state is generated, and thereby a 5-coordination structure is developed. In addition to oxides (oxo complexes) such as γ-alumina, even in amine complexes, the coordination unsaturation structure is believed to be developed. Whether or not aluminum develops a 5-coordination structure can be determined through 27Al-NMR measurement. The peak appearing at 0 to 10 ppm in the obtained NMR chart shows a 6-coordination structure; the peak appearing at 25 to 35 ppm shows a 5-coordination structure; and the peak appearing at 25 to 35 ppm shows a 4-coordination structure. Therefore, the abundance of the aluminum having each coordination structure can be calculated from the ratio among the peak areas in 27Al-NMR chart. The aluminum having a 5-coordination structure has a proper acid-basic strength and activates both oxygen of alcohol (ROH) and oxygen of alkylene oxide. Therefore, if the catalyst contains such aluminum, polyalkylene glycols can be effectively produced. In contrast, if the catalyst contains no aluminum having a 5-coordination structure, the catalyst is not proper acid. Therefore, the ROH or the alkylene oxide is not properly activated, which fails to exhibit the functional effects effectively.

In the above-mentioned aluminum compound, the peak area at 25 to 35 ppm more preferably accounts for 50% or more and still more preferably 54% or more, relative to a total of the peak areas at 0 to 10, 25 to 35 and 45 to 55 ppm.

Measurement conditions of the above-mentioned 27Al-NMR are preferably as follows. In the present description, the measurement conditions of 27Al-NMR are the same as the following conditions.
Device: product of JEOL Ltd., ECA600, magnet 14.1T
Measurement condition: a sample is charged into a 4 mm zirconia rotor tube, MAS (spinning) 18 kHz, room temperature resonant frequency 156.39 MHz, DD/MAS measuring method, pulse width 1.2 microseconds, repeat time 2 seconds, integration times 2000 to 30000 times (different depending on samples)
Peak-resolution method: software for measurement (DELTA) Gauss Lorentz function is used.

The above-mentioned aluminum compound preferably contains aluminum and at least one element belonging to 3 to 15 Groups. Examples of the above-mentioned at least one element belonging to 3 to 15 Groups include scandium, titanium, zirconium, hafnium, vanadium, chromium, manganese, iron, cobalt, nickel, copper, zinc, boron, gallium, silicon, and germanium. It is particularly preferable the above-mentioned at least one element belonging to 3 to 15 Groups is at least one element selected from titanium, zirconium, hafnium, boron, gallium, silicon, and germanium. The aluminum compound containing these elements can exhibit excellent activity as the catalyst and therefore can effectively produce a polyalkylene glycol in the production of polyalkylene glycols. The above-mentioned at least one element belonging to 3 to 15 Group is preferably silicon.

The above-mentioned aluminum compound is preferably produced by carrying aluminum on the silica surface. The amount of the carried aluminum after the treatment is preferably 0.5 to 40% relative to the total weight of the aluminum compound, and more preferably 0.5 to 10%, and still more preferably 0.5 to 5%.

The aluminum compound containing these elements can exhibit excellent activity as the catalyst and therefore produce a polyalkylene glycol effectively in the production of polyalkylene glycols. In this case, it is preferable in the 27Al-NMR measurement that the peak area having the maximum value at 25 to 35 ppm accounts for 40% or more relative to a total of the peak areas at 0 to 10, 25 to 35, and 45 to 55 ppm.

The above-mentioned “carrying” means that a hydroxyl group on the silica surface is thermally or chemically converted and bonded to an aluminum atom with an oxygen atom therebetween. Thus, if the aluminum is carried on the silica surface, a 5-coordination structure is believed to develop in the aluminum.

It is preferable that the above-mentioned aluminum compound is bonded to at least one element selected from nitrogen, phosphorus, oxygen, and sulfur. The aluminum compound bonded to these elements can exhibit excellent activity as the catalyst and therefore can produce a polyalkylene glycol effectively in the production of polyalkylene glycols.

The preferable embodiments of the present invention include an embodiment in which, in the above-mentioned production method of polyalkylene glycols, polyalkylene glycols produced by the above-mentioned production method are polymerizable polyalkylene glycols satisfying the following characteristics (1) and (2): (1) a polymerizable dimer content is 0.05 to 10% by weight; and (2) a metal content is 50 ppm or less.

With respect to the physical properties of the polyalkylene glycols produced by the above-mentioned production method, it is preferable that such polyalkylene glycols satisfy characteristics of polyalkylene glycols mentioned below.

The reaction-system is not especially limited in the above-mentioned production method and may be a slurry-bed or a fixed bed reaction system. The reaction system may be appropriately determined depending an productivity. The use amount of the catalyst is not especially limited. The catalyst is preferably 0.1 to 50% by weight relative to the raw material unsaturated initiator, and more preferably 1 to 50% by weight, and still more preferably 3 to 30% by weight.

The unsaturated initiator in the present invention is a compound with which an alkylene oxide can be additionally reacted. The unsaturated initiator is particularly preferably an active hydrogen compound. Compounds containing a hydroxyl group may be mentioned as the active hydrogen compound, for example.

It is preferable that the unsaturated initiator further contains an unsaturated group at the terminal. The preferable embodiment of the present invention include an embodiment in which the production method of polyalkylene glycols, wherein the unsaturated initiator is an unsaturated initiator having a terminal unsaturated group. Use of the unsaturated initiator having a terminal unsaturated group makes it possible for polyalkylene glycols produced to have an unsaturated group derived from the unsaturated initiator in the terminal structure. Polymerizable polyalkylene glycols having an unsaturated monomer are useful as a polymerization monomer.

The following compounds (I) to (IX) are preferable as the above-mentioned unsaturated initiator.
(I) Hydroxyalkyl maleate or hydroxyalkyl fumarate represented by the following formula (1):
   HO—Xm—OCO—CH═CH—COO—Xn—OH (1)

(in the formula, Xm being an alkyl group having a straight or branched chain containing 0 to 18 carbon atoms; Xn being an alkyl group having a straight or branched chain containing 1 to 18 carbon atoms.).

In the above formula (1), the carbon numbers in Xm and Xn are the same or different and more preferably 1 to 10, and still more preferably 2 to 5.
(II) Diols represented by the following formula (2) or (3):
   HO—Xp—CH═CH—Xq—OH (2)

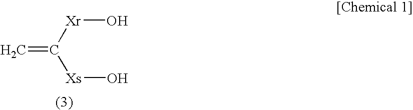

(in the formulae (2) and (3), Xp, Xq, Xr, and Xs being the same or different and alkyl groups having a straight or branched chain containing 1 to 18 carbon atoms.).

In the above formulae (2) and (3), the carbon numbers in Xp, Xq, Xr, and Xs are the same or different and more preferably 1 to 10, and still more preferably 2 to 5.
(III) Hydroxyalkyl itaconates represented by the following formula (4):

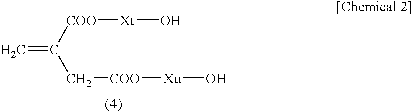

(in the formula, Xt and Xu being the same or different and alkyl groups having a straight or branched chain containing 0 to 18 carbon atoms, and the carbon numbers in Xt and Xu being not 0.).

In the above formula (4), the carbon numbers in Xt and Xu are the same or different and more preferably 1 to 10, and still more preferably 2 to 5.
(IV) Alkenyl alcohols represented by the following formula (5):

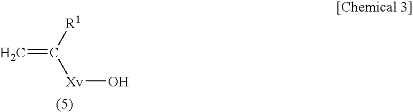

(in the formula, R¹, representing a hydrogen atom or a methyl group, Xv being an alkyl group having a straight or branched chain containing 1 to 18 carbon atoms.).

In the above-mentioned formula (5), the carbon number in Xv is more preferably 1 to 10, and still more preferably 2 to 5.
(V) Hydroxyalkyl (meth)acrylates represented by the following formula (6):

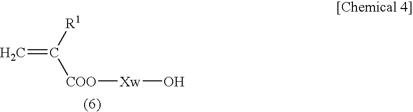

(in the formula, R²representing a hydrogen atom or a methyl group, Xw being an alkyl group having a straight or branched chain containing 2 to 18 carbon atoms.).

In the above formula (6), the carbon number in Xw is more preferably 2 to 10, and still more preferably 2 to 5.
(VI) The following formula (7):

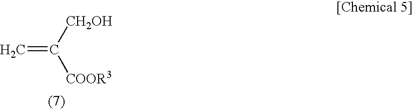

(in the formula, R³ representing a hydrogen atom or an alkyl group having a straight or branched chain containing 1 to 20 carbon atoms.).
(VII) Hydroxystyrene compounds represented by the following formula (8):
   CH₂═C—C₆H₍₅₋ₓ₎—(OH)ₓ (8)

(in the formula, x being an integer of 1 to 3.).

Phenylene part-containing unsaturated initiators typified by the following formulae (9) and (10):
CH₂═CH—C₆H₄—Xy—OH (9)
CH₂═CH—Xz—C₆H_{d}—OH (10)

(in the formulae (9) and (10), Xy and Xz being the same or different and alkyl groups having a straight or branched chain containing 1 to 18 carbon atoms.).
(IX) Alkylene oxide adducts of the above-mentioned compounds represented by the above formulae (1) to (10).

As mentioned above, the active hydrogen compounds containing an unsaturated group are preferably used as the unsaturated initiator used in the present invention. Active hydrogen compounds containing a terminal unsaturated group are more preferred. Hydroxyalkyl compounds having a terminal unsaturated group are still more preferable. Hydroxyalkyl esters containing a (meth)alkyl alcohol and a terminal unsaturated group are particularly preferable. Hydroxyalkyl (meth)acrylates are most preferable. Among them, hydroxyethyl (meth)acrylate and hydroxypropyl (meth)acrylate are most preferable.

Alkylene oxides containing 2 to 18 carbon atoms or styrene oxide are preferable as the alkylene oxide used in the above-mentioned reaction. These oxides may be used singly or in combination of two or more species of them. The carbon number is preferably 2 to 18, and more preferably 2 to 8, and still more preferably 2 to 4. Specific examples of such oxides include ethylene oxide, propylene oxide, butylene oxide, and styrene oxide. Any two or more alkylene oxide adducts may be added randomly, alternatively, or in block. The term “alkylene oxide”in the present description means alkylene oxides including styrene oxide.

The average molar number of addition of an oxyalkylene group or an oxystyrene group in this reaction is suitably 1 to 300, and preferably 1 to 110, and more preferably 1 to 50, and still more preferably 1 to 30, and particularly preferably 1 to 25, and most preferably 1 to 20, and still more preferably 1 to 10, and still more preferably 1 to 5.

Thus, the preferable embodiments of the present invention include an embodiment in which the above-mentioned polymerizable polyalkylene glycol is produced by reacting the alkylene oxide with the unsaturated initiator, and the average molar number of addition of the above-mentioned alkylene oxide is 1 to 300.

Then, a general embodiment of the method for producing a polyalkylene glycol by reacting the alkylene oxide with the unsaturated initiator in the above-mentioned production method is mentioned below. The present invention is not limited to only this embodiment.

In the above-mentioned production method, the following embodiment is generally performed, for example. The catalyst, the unsaturated initiator, and the alkylene oxide are appropriately charged into a reactor and thereby reaction is allowed to proceed, and the reaction is completed at the time when the amount of the alkylene oxide remaining in the reactor reaches a predetermined concentration or less. The above-mentioned reaction is exothermic reaction. The reaction is started at the time when the unsaturated initiator and the alkylene oxide are coexistent in the presence of the catalyst. The reaction is completed by lowering the temperature of the reaction solution than a predetermined reaction temperature by cooling and the like.

The method (order) of charging the above-mentioned unsaturated initiator and alkylene oxide is not especially limited. For example, part or all of the unsaturated initiator is initially charged into a reactor and thereto the alkylene oxide and the rest of the unsaturated initiator are added. Part or all of the alkylene oxide may be initially charged.

The above-mentioned addition method of the unsaturated initiator and the alkylene oxide may be one step-charge or successive charge (continuous charge and/or intermittent charge). The initially charged matter is preferably charged in one step and the rest of the charged matter is preferably charged successively. The continuous charge means an embodiment in which the unsaturated initiator and the alkylene oxide are continuously charged little by little. The intermittent charge means an embodiment in which the unsaturated initiator and the alkylene oxide are charged in any portions with a pulse interval or intermittently. In the continuous charge, the charge may be allowed to proceed at a certain charge rate until completion of the charge or the charge may be allowed to proceed while the charge rate itself is arbitrarily changed continuously. It is preferable that the charge rate is lowered from before to after the change if changed during the charge.

The unsaturated initiator and the alkylene oxide may be added from separate charge lines, or previously mixed with each other in a piping, a line mixer, a mixing tank, or the like before charged into a reactor, if charged simultaneously. However, in the addition from separate charge lines, the molar ratio of the alkylene oxide to the unsaturated initiator in the system may not be balanced. Therefore, it is preferable that they are previously mixed with each other before charged into a reactor and then added. In the addition from separate charge lines, the charge forms (one-step charge, successive charge), the temperatures of the raw materials charged, the charge rates, and the like may not be necessarily the same.

With respect to the temperature when the above-mentioned unsaturated initiator and alkylene oxide are charged, they may be charged at normal temperatures, or previously heated to a desired temperature and then charged in order not to change the temperature inside the system at that time. The time required until completion of the charge of the total supply amount of the above-mentioned unsaturated initiator and alkylene oxide is not especially limited and may be appropriately determined in consideration of proceeding of the reaction, productivity, and the like.

In the above-mentioned reaction of the unsaturated initiator with the alkylene oxide, a polymerization inhibitor may be added into the reaction system, if necessary. The polymerization inhibitor is not especially limited as long as it is a generally industrially used polymerization inhibitor. One or two or more species of the following compounds may be used. Phenolic compounds such as hydroquinone, methylhydroquinone, tert-butyl hydroquinone, 2,6-di-tert-butyl hydroquinone, 2,5-di-tert-butyl hydroquinone, 2,4-dimethyl-6-tert-butyl phenol, and hydroquinone monomethyl ether; p-phenylenediamines such as N-isopropyl-N′-phenyl-p-phenylenediamine, N-(1,3-dimethylbutyl)-N′-phenyl-p-phenylenediamine, N-(1-methylhebutyl)-N′-phenyl-p-phenylenediamine, N,N′-diphenyl-p-phenylenediamine, and N,N′-di-2-naphthyl-p-phenylenediamine; amine compounds such as thiodiphenylamine and phenothiazine; copper dialkyldithiocarbamates such as copper dibutyldithiocarbamate, copper diethyldithiocarbamate, and copper dimethyldithiocarbamate; N-oxyl compounds such as 2,2,4,4-tetramethylazetidine-1-oxyl, 2,2-dimethyl-4,4-dipropylazetidine-1-oxyl, 2,2,5,5-tetramethylpyrrolidine-1-oxyl, 2,2,5,5-tetramethyl-3-oxopyrrolidine-1-oxyl, 2,2,6,6-tetramethylpiperidine-1-oxyl, 4-hydroxy-2,2,6,6,-tetramethylpiperidine-1-oxyl, 6-aza-7,7-dimethyl-spiro(4,5)decane-6-oxyl, 2,2,6,6-tetramethyl-4-acetoxypiperidine-1-oxyl, 2,2,6,6-tetramethyl-4{benzoyloxypiperidine-1-oxyl, and 4,4′,4″-tris(2,2,6,6-tetramethylpiperidine-1-oxyl) phosphate.

The addition amount of the above-mentioned polymerization inhibitor is preferably 0.0001 to 1% by weight relative to 100% by weight of the total supply amount of the raw material unsaturated initiator. The addition amount is more preferably 0.001 to 0.5% by weight. The addition timing of the polymerization inhibitor is not especially limited. It is preferable that the polymerization inhibitor is initially charged in the reactor together with the initially charged components.

In the above-mentioned reaction, a solvent maybe existent if necessary to perform the reaction, for moderate proceeding of the reaction. One or two or more species of common solvents such as toluene, xylene, heptane, and octane may be used as the solvent.

The reaction temperature of the above-mentioned unsaturated initiator and alkylene oxide is preferably 30 to 160° C., generally. If the reaction temperature is less than 30° C., the reaction rate is remarkably lowered, and thereby the productivity is reduced. It the reaction temperature is more than 160° C., increase of byproducts such as diesters or polymerization of the unsaturated initiator may be caused. The reaction temperature is more preferably 30 to 120° C., and still more preferably 40 to 110° C., and particularly preferably 40 to 100° C. The pressure inside the reactor at the above-mentioned reaction is preferably pressurization although it depends on the kind of the raw materials used and the use proportion thereof.

The termination timing of the above-mentioned reaction (in other words, cooling starting timing of the reaction) can be determined based on the time when the residual alkylene oxides have disappeared enough. The time when the residual alkylene oxide have disappeared enough means the time when the concentration of the alkylene oxides has reached a concentration enough not to cause problems in terms of safety and productivity.

Polyalkylene glycols obtained by the above-mentioned method are useful as a raw material of polymers used in various applications because such glycols have an unsaturated group in the molecule. Such polyalkcylene glycols can be used as various polymer materials, cohesive agents, adhesive agents, coating materials, cosmetics additives, concrete admixtures, cement dispersants, cleaning agents, clay dispersants, various dispersants such as metal dispersants for electron material abrading agents.

Then, the method of polymerizing a monomer component containing the above-mentioned polyalkylene glycol and thereby producing a (co)polymer ((co)polymer product) is mentioned. In such a method, only the above-mentioned monomer may be polymerized, or the above-mentioned monomer and a monomer polymerizable with the above-mentioned monomer may be copolymerized. The upper limit of the above-mentioned polyalkylene glycol in 100% by weight of the total monomer component is preferably 99% by weight, and more preferably 57% by weight, and still more preferably 95% by weight, and particularly preferably 90% by weight, and most preferably 80% by weight.

The lower limit is preferably 1% by weight, and more preferably 5% by weight, and still more preferably 10% by weight, and particularly preferably 20% by weight, and most preferably 40% by weight.

The above-mentioned monomer copolymerizable with the polyalkylene glycol is not especially limited. Maleic acid and derivatives thereof may be mentioned. One or two or more species of them may be used. The derivatives of maleic acid are not especially limited. Examples thereof include maleic anhydride; half esters of maleic acid with alcohols containing 1 to 30 carbon atoms; half amides of maleic acid with amines containing 1 to 30 carbon atoms; half amides or half esters of maleic acid with amino alcohols containing 1 to 30 carbon atoms; half esters of maleic acid with compounds in which an average 1 to 500 mol of alkylene oxides is added to alcohols containing 1 to 30 carbon atoms; half amides of maleic acid with compounds prepared by aminodating an hydroxyl group at one terminal of compounds in which an average 1 to 500 mol of alkylene oxides is added to alcohols containing 1 to 30 carbon atoms; half asters of maleic 30 acid with glycols containing 2 to 18 carbon atoms or polyalkylene glycols in which an average 2 to 500 mol of such glycols are added; half amides of maleamine acid and glycols containing 2 to 18 carbon atoms or poyalkylene glycols in which an average 2 to 500 mol of such glycols are added; and monovalent metals thereof, divalent metals thereof, ammonium salts thereof, and organic ammonium salts thereof. Alkali metals such as sodium and potassium are preferred as the monovalent metals. Alkaline earth metals such as calcium and magnesium are preferable as the divalent metals The organic ammonium is a protonated organic amine. Preferred examples thereof include alkanol ammonium such as ethanol ammonium, diethanol ammonium, and triethanol ammonium; and alkyl ammonium such as triethyl ammonium. Among them, it is preferable that the monomer component essentially contains at least one monomer selected from the group consisting of maleic acid and salts thereof, maleic anhydride, and maleate. It is particularly preferable that the monomer component essentially contains maleic anhydride or maleic acid.

The following monomers may be mentioned as the monomer copolymerizable with the polyalkylene glycol of the present invention, in addition to maleic acid.

Unsaturated monocarboxylic acids such as acrylic acid, methacrylic acid, crotonic acid and monovalent metal salts thereof, divalent metal salts thereof, ammonium salts thereof, organic ammonium salts thereof; unsaturated dicarboxylic acids such as fumaric acid, itaconic acid, and citraconic acid, and monovalent metal salts thereof, divalent metal salts thereof, ammonium salts thereof, organic ammonium salts thereof; half esters or diesters of unsaturated dicarboxylic acids such as umaric acid, itaconic acid, and citraconic acid, with alcohols containing 1 to 30 carbon atoms; half amides or diamides of the above-mentioned unsaturated dicarboxylic acids with amines containing 1 to 30 carbon atoms; half esters or diesters of the above-mentioned unsaturated dicarboxylic acids with alkyl (poly)alkylene glycols in which 1 to 500 mol of alkylene oxides containing 2 to 18 carbon atoms are added to the above-mentioned alcohols or amines; half esters or diesters of the above-mentioned unsaturated dicarboxylic acids with glycols containing 2 to 18 carbon atoms or polyalkylene glycols in which 2 to 500 mol of these glycols are added; esters of alcohols containing 1 to 30 carbon atoms with unsaturated monocarboxylic acids such as methyl (meth)acrylate, ethyl (meth)acrylate, propyl (meth)acrylate, glycidyl (meth)acrylate, methyl crotonate, ethyl crotonate, and propyl crotonate; esters of unsaturated monocarboxylates such as (meth)acrylic acid with alkoxy(poly)alkylene glycols in which 1 to 500 mol of alkylene oxides containing 2 to 18 carbon atoms are added to alcohols containing 1 to 30 carbon atoms; and adducts prepared by 1 to 500 mol of alkylene oxides containing 2 to 18 carbon atoms to unsaturated monocarboxylic acids such as (meth)acrylic acid, such as (poly)ethylene glycol monomethacrylate, (poly)propylene glycol monomethacrylate, and (poly)butylene glycol monomethacrylate.

(Poly)alkylene glycol di(meth)acrylates such as triethylene glycol di(meth)acrylate, (poly)ethylene glycol di(meth)acrylate, polypropylene glycol di(meth)acrylate, and (poly)ethylene glycol (poly)propylene glycol di(meth)acrylate; polyfunctional (meth)acrylates such as hexanediol di(meth)acrylate, trimethylolpropane tri(meth)acrylate, and trimethylolpropane di(meth)acrylate; (poly)alkylene glycol dimaleates such as triethylene glycol dimaleate and polyethylene glycol dimaleate; unsaturated sulfonic acids such as vinyl sulfonate, (meth)alkyl sultonate, 2-(meth)acryloxy ethyl sulfonate, 3-(meth)acryloxy propyl sulfonate, 3-(meth)acryloxy-2-hydroxypropyl sulfonate, 3-(meth)acryloxy-2-hydroxypropyl sulfophenyl ether, 3-(meth)acryloxy-2-hydroxypropyloxysulfobenzoate, 4-(meth)acryloxy butylsulfonate, (meth)acrylamide methylsulfonate, (meth)acrylamide ethylsulfonate, 2-methylpropane sulfonate (meth)acrylamide, and styrene sulfonate, and monovalent metal salts thereof, divalent metal salts thereof, ammonium salts thereof, and organic ammonium salts thereof; amides of unsaturated monocarboxylic acids with amines containing 1 to 30 carbon atoms, such as methyl (meth)acrylamide; vinyl aromatic groups such as styrene, a-methyl styrene, vinyltoluene, and p-methyl styrene; alkanediol mono(meth)acrylates such as 1,4-butanediol mono(meth)acrylate, 1,5-pentanediol mono(meth)acrylate, and 1,6-hexanediol mono(meth)acrylate; dienes such as butadiene, isoprene, 2-methyl-1,3-butadiene, and 2-chloro-1,3-butadiene.

Unsaturated amides such as (meth)acrylamide, (meth)acryl alkylamide, N-methylol (meth)acrylamide, and N,N-dimethyl (meth)acrylamide; unsaturated cyano compounds such as (meth)acrylonitrile, and α-chloroacrylonitrile: unsaturated esters such as vinyl acetate, and vinyl propionate; unsaturated amines such as aminoethyl (meth)acrylate, methylaminoethyl (meth)acrylate, dimethylaminoethyl (meth)acrylate, dimethylaminopropyl (meth)acrylate, dibuthylaminoethyl (meth)acrylate, and vinylpyridine; divinyl aromatic groups such as divinyl benzene; cyanurates such as trialkyl cyanurate; alkyl compounds such as (meth)alkyl alcohol and glycidyl (meth)alkyl ether; siloxane derivatives such as polydimethyl siloxane propyl amino maleamic acid, polydimethyl siloxane aminopropylene amino maleamic acid, polydimethyl siloxane-bis-(propyl amino maleamic acid), polydimethyl siloxane-bis-(dipropylene amino maleamic acid, polydimethyl siloxane-(1-propyl-3-acrylate), polydimethyl siloxane-(1-propyl-3-methacrylate), polydimethyl siloxane-bis-(1-propyl-3-acrylate), and polydimethyl siloxane-bis-(1-propyl-3-methacrylate).

N-vinyl compounds suck as N-vinylsuccucinimide, N-vinylcarbazole, 1-vinylimidazole, N-vinylcaprolactam, N-vinyloxazolidone, N-vinylpyrrolidone, N-vinylformamide, N-methyl-N-vinylformamide, N-vinylacetamide, and N-methyl-N-vinylacetamide.

and vinyl ethers such as methyl vinyl ether, ethyl vinyl ether, 2-hydroxyethyl vinyl ether, 4-hydroxybutyl vinyl ether, and diethylene glycol monovinyl ether.

In the above-mentioned polymerization method of the polyalkylene glycol, the monomer component is preferably polymerized using a polymerization initiator. The polymerization can be performed by solvent polymerization, bulk polymerization, and the like. The solution polymerization can be performed in batch-wise system or continuous system. The solvent used in such polymerization is not especially limited. Examples of such a solvent include water; alcohols such as methyl alcohol, ethyl alcohol, and isopropyl alcohol; aromatic or aliphatic hydrocarbons such as benzene, toluene, xylene, cyclohexane, and n-hexane; ester compounds such as ethyl acetate; ketone compounds such as acetone and methyl ethyl ketone; and cyclic ether compounds such as tetrahydrofuran and dioxane. If maleic anhydride is used as the maleic acid monomer, the following inert solvents are preferably used for prevention of cleavage of the acid anhydride group. Aromatic or aliphatic hydrocarbons such as benzene, toluene, xine, cyclohexane, and n-hexane; ester compounds such as ethyl acetate; ketone compounds such as acetone and methyl ethyl ketone are preferably used. In contrast, if maleic acid (salt) and the like is used as the maleic acid monomer, at least one selected from the group consisting of water and lower alcohols containing 1 to 4 carbon atoms is preferably used. Among them, water is more preferably used because a solvent-removing step can be omitted.

In the above-mentioned aqueous solution polymerization, water-soluble polymerization initiators maybe used as a radical polymerization initiator. Examples of such water-soluble polymerization initiators include persulfates such as ammonium persulfate, sodium persulfate, and potassium persulfate; hydrogen peroxide; and water-soluble azo initiators, such as azoamidine compounds such as 2,2′-azobis-2-methylpropioneamidine hydrochloride, cyclic azoamidine compounds such as 2,2′-azobis-2-(2-imidazoline-2-yl)propane hydrochloride, and azonitrile compounds such as 2-carbamoylazoisobutyronitrile. In this case, the following accelerators (reducing agents) may be used in combination. Alkali metal sulfites such as sodium hydrogensulfite, metanisulfite, sodium hypophosphite, Fe(II) salts such as Mohr's salt, sodium hydroxy methane sulfinate dihydrate, hydroxylamine hydrochloride, thiourea, L-ascorbic acid (salt), and erythorbic acid (salt). Among them, combinations of hydrogen peroxide and organic reducing agents are preferred. Preferred examples of organic reducing agents include L-ascorbic acid (salt), L-ascorbate, erythorbic acid (salt), and erythorbate. These radical polymerization initiators or accelerators (reducing agents) may be used singly or in combination of two or more species of them.

If lower alcohols, aromatic or aliphatic hydrocarbons, ester compounds or ketone compounds are used as the solvent to perform the solution polymerization, or if the bulk polymerization is performed, peroxides such as benzoyl peroxide, lauroyl peroxide, and sodium peroxide; hydroperoxides such as t-butyl hydroperoxide and cumene hydroperoxide; and azo compounds such as azobisisobutyronitrile maybe used as a radical polymerization initiator. In this case, accelerators such as amine compounds may be used in combination. If water-lower alcohol mixed solvents are used, such solvents may be appropriately selected from the above-mentioned radical polymerization initiators or combinations of the radical polymerization initiators and the accelerators. The polymerization temperature may be appropriately determined depending on the solvent used or the polymerization initiator used. The polymerization temperature is usually within a range of 0 to 150° C.

The method of charging each of the monomers into a reactor is not especially limited. Examples of the method include a method of charging the total amount of the monomers into the reactor in one step in early stages; a method of charging the total amount of the monomers into the reactor in portions or continuously; a method of charging part of the monomers into the reactor in early stages, and then changing the rest of the monomers into the reactor in portions or continuously. The radical polymerization initiator may be initially charged or added dropwise into the reactor. These methods may be combined depending on the purpose.

In the above-mentioned polymerization, a chain transfer agent may be used in order to adjust the molecular weight of the obtained (co)polymer. The chain transfer agent is not especially limited. Examples of such a chain transfer agent include conventionally known hydrophilic chain transfer agents, for example, thiol chain transfer agents such as mercaptoethanol, thioglycerol, thioglycolic acid, 3-mercaptopropionic acid, thiomalic acid, and 2-mercaptoethanesulfonic acid; secondary alcohols such as isopropyl alcohol; and lower oxides and salts thereof, such as phosphorous acid, phosphinic acid and salts thereof (sodium hypophosphite, potassium hypophosphite, and the like), sulfurous acid, hydrogen sulfite, dithionic acid, and metabisulfurous acid and salts thereof (sodium sulfite, sodium hydrogensulfite, sodium dithionite, sodium metabisulfite, and the like). Use of hydrophobic chain transfer agents is effective in improvement in viscosity of concrete compositions. Preferred examples of such hydrophobic chain transfer agents include thiol chain transfer agents having a hydrocarbon group containing 3 or more carbon atoms, such as butanethiol, octanethiol, decanethiol, dodecanethiol, hexadecanethiol, octadecanethiol, cyclohexyl mercaptan, thiophenol, octyl thioglycolate, and octyl 3-mercaptopropionate. Two or more chain transfer agents may be in combination. Hydrophilic chain transfer agents and hydrophobic chain transfer agents may be used in combination. Monomers with high chain transfer property such as (meth)alkyl sulfonic acid (salt) are effectively used as the monomer component, in order to adjust the molecular weight of the (co)polymer.

In the above-mentioned polymerization, the polymerization reaction needs to proceed stably for production of copolymers having a predetermined molecular weight with good reproducibility. Therefore, it is preferable in the solution polymerization that the dissolved oxygen concentration at 25° C. of the solvent used is within a range of 5 ppm or less. The dissolved oxygen concentration is more preferably within a range of 0.01 to 4 ppm, and still more preferably within a range of 0.01 to 2 ppm, and most preferably within a range of 0.01 to 1 ppm. If the monomer is added to the solvent and then nitrogen substitution is performed, the dissolved oxygen concentration in the system containing also the monomer is preferably determined within the above-mentioned range.

The above-mentioned dissolved oxygen concentration of the solvent may be adjusted in a polymerization reactor. Solvents in which the dissolved oxygen concentration is previously adjusted may be used. The following (1) to (5) methods may be mentioned as the method of removing oxygen in the solvent.
(1) Inert gas such as nitrogen is charged, under pressurization, into a sealed container into which a solvent is charged. Then, the pressure inside the sealed container is lowered to lower partial pressure of oxygen in the solvent. The pressure inside the sealed container may be lowered in nitrogen air.
(2) A liquid phase is churned for a long time while a gas phase inside a container in which a solvent is charged is substituted with inert gas such as nitrogen.
(3) Inert gas such as nitrogen is bubble into a solvent charged in a container, for a long time.
(4) A solvent is once boiled and then cooled under nitrogen gas atmosphere such as nitrogen.
(5) A static mixer is located in a piping, and a solvent and inert gas such as nitrogen are mixed inside the piping for transferring the solvent into a polymerization reactor.

(Co)polymers obtained by the above-mentioned polymerization method are used as a main component of a dispersant and the like, as they are. The pH range of the (co)polymer may be adjusted if necessary, and then used. It is preferable in terms of handling that the pH range is adjusted to weak acidity or more in the aqueous solution state. The pH is more preferably 4 or more, and still more preferably 5 or more, and particularly preferably 6 or gore. In addition, the copolymerization reaction in the aqueous solution may be performed under pH 7 or more. In such a case, the polymerization degree may be reduced and at the same time, the copolymerizability get worse and the dispersibility is lowered. Therefore, the copolymerization reaction is preferably performed within a pH range of acid to neutral. The pH is more preferably less than 6, and still more preferably less than 5.5, and particularly preferably less than 5. Accordingly, it is preferable that the polymerization reaction is performed under a low pH, and then an alkaline substance is added to adjust the pH to higher pH. As preferable embodiments, the following methods may be mentioned, for example. Method of performing the copolymerization reaction under less than pH 6, and then an alkaline substance is added to adjust the pH to 6 or more; method of performing the copolymerization reaction under less than pH 5, and then an alkaline substance is added to adjust the pH to 5 or more; and method of performing the copolymerization reaction under less than pH 5, and then an alkaline substance is added to adjust the pH to 6 or more. The pH may be adjusted using inorganic salts such as hydroxides or carbonates of monovalent metals or divalent metals; ammonia; and alkaline substances such as organic amines. If the pH needs to be lowered, particularly if the pH needs to be adjusted at the polymerization, the pH may be adjusted with acid substances such as phosphoric acid, sulfuric acid, nitric acid, alkyl phosphoric acid, alkyl sulfuric acid, alkyl sulfonic acid, and (alkyl)benzenesulfonic acid. Among these acid substances, phosphoric acid is preferred because phosphoric acid has a pH buffer action. After completion of the reaction, the concentration adjustment may be performed, if necessary.

The weight average molecular weight of the above-mentioned (co)polymer is not especially limited. If the (co)polymer is used as a main component of a dispersant, for example, the weight average molecular weight on polyethylene glycol equivalent basis, determined by gel permeation chromatography (hereinafter, “GPC”), is preferably 1000 to 500000, and more preferably 5000 to 300000, and still more preferably 10000 to 150000. If such a weight average molecular weight range is selected, higher dispersibility is exhibited.

The above-mentioned (co)polymer may contain an antifoaming agent if used in various applications such as dispersant. In this case, the antifoaming agent may be added after the production of the above-mentioned polymer, or may be added before or during the polymerization. The addition proportion is preferably 0.0001 to 10% by weight relative to 100% by weight of the total amount of the polymer.

One or two or more compounds may be used as the above-mentioned antifoaming agent, for example. Polyoxyalkylenes such as (poly)oxyethylene (poly)oxypropylene adduct; polyoxyalkylene alkyl ethers such as diethylene glycol heptyl ether, polyoxyethylene oleyl ether, polyoxypropylene butyl ether, polyoxyethylene polyoxypropylene 2-ethylhexyl ether, oxyethylene oxypropylene adduct to higher alcohols containing 12 to 14 carbon atoms; polyoxyalkylene (alkyl)alkyl ethers such as polyoxypropylene phenyl ether, polyoxyethylene nonylphenyl ether; acetylene ethers prepared by addition polymerization of alkylene oxides with acetylene alcohols, such as 2,4,7,9-tetramethyl-5-desine-4,7-diol, 2,5-dimethyl-3-hexyne-2,5-diol, and 3-methyl-1-butyne-3-ol; (poly)oxyalkylene fatty acid esters such as diethylene glycol oleate, diethylene glycol laurate and ethylene glycol distearate; polyoxyalkylene sorbitan fatty acid esters such as polyoxyethylene sorbitan monolaurate and polyoxyethylene sorbitan trioleate; polyoxyalkylene alkyl (aryl)ether sulfates such as polyoxypropylene methyl ether sodium sulfate and polyoxyethylene dodecyl phenyl ether sodium sulfate; polyoxyalkylene alkyl phosphates such as polyoxyethylene stearylphosphate; polyoxyalkylene alkylamines such as polyoxypropylene polyoxyethylene laurylamine (1 to 20 mol of propylene oxide is added, ethylene oxide 1 to 20 mol adduct) and hardened beef tallow amine to which an alkylene oxide is added (1 to 20 mol of propylene oxide is added, ethylene oxide 1 to 20 mol adduct); and polyoxyalkylene amides.

If the above-mentioned (co)polymer is used, the (co)polymer may be used in an aqueous solution form, or in a powder form prepared by the following procedures. After the polymerization, the (co)polymer is neutralized with a hydroxide of a divalent metal such as calcium and magnesium to be a polyvalent metal salt, and then dried; or carried on an inorganic powder such as silica fine particles and then dried; or dried or solidified to be a thin film on a support using a drum drier, a disk drier, or belt drier, and then pulverized.

The polyalkylene glycols of the present invention satisfy the above-mentioned characteristics (1) and/or (2), and preferably satisfies both of the above-mentioned characteristics (1) and (2). Accordingly, the preferable embodiments of the present invention include an embodiment in which the above-mentioned polymerizable polyalkylene glycol satisfies the above-mentioned characteristics (1) and (2).

The polyalkylene glycols of the present invention are characterized in that the above-mentioned solid acid catalyst is used to perform reaction and the catalyst is separated to produce polyalkylene glycol mono (meth)acrylates with high purity. Further, the polyalkylene glycols of the present invention are characterized in that polyalkylene glycol mono(meth)acrylates with high purity are obtained from residues obtained by distillation. The following effects can be obtained in the polyalkylene glycols of the present invention because the solid catalyst is used. (1) catalysts usually containing metals can be easily separated from the reaction solution by a simple method such as filtration and centrifugal separation. (2) Therefore, no addition of water or organic solvents is needed for extraction, and no removal of such solvents for extraction by a method such as distillation is needed. (3) Generally, catalysts containing metals are heated and there by modification in desired products may be generated. However, the catalysts can be easily removed from the reaction solution, as mentioned above. Therefore, desired products with high purity can be produced. The “modification of desired products” used herein means generation, discoloration, and coloring in oxides, which are caused by oxidation reaction; generation of polymerizable dimers caused by disproportionation; or generation of polymers. It is preferable in the polyalkylene glycols of the present invention that the above-mentioned metal content is 50 ppm or less. The metal content is more preferably 10 ppm or less, and still more preferably 100 ppb or less. If the metal content is reduced as mentioned above, generation of peroxides can be suppressed. Therefore, the polymerization proceeds stably and high molecular weight polymers can be obtained.

In addition, it is preferable that the hue (APHA value) Hazen is 40 or less. In the above-mentioned hue, the Hazen is preferably 5 to 35, and more preferably 10 to 20.

If the hue (APHA value) is reduced as mentioned above, coloring of resins can be reduced enough. Therefore, such resins can be preferably used as a resin for optical materials mentioned below and useful in applications such as white cement. The preferable embodiments of the present invention include an embodiment in which the polymerizable polyalkylene glycol has a hue (APHA value) of 1 to 40.

The hue can be measured based on the method of color test of chemicals-Hazen Color Number (platinum-cobalt scale) according to JTS K 0071-1 (1998). The APHA value is a value based on this standard. The above-mentioned metal content can be measured by a commercially available high frequency inductively coupled plasma (ICP) analysis apparatus.

The above-mentioned polymerizable dimer means a crosslinking compound having two or more double bonds in the molecule. If the above-mentioned polymerizable dimer is contained in the above-mentioned polyalkylene glycol as an impurity, a crosslinking structure is formed when the polyalkylene glycol is polymerized, and gelling is easily occurred, which may cause reduction in dispersibility.

Therefore, among the above-mentioned crosslinking compounds having two or more double bonds in the molecule, particularly crosslinking compounds having one double bond at each terminal of the molecule need to be reduced. Diesters and di(alkyl)ethers may be mentioned, for example, as the above-mentioned crosslinking compounds having one double bond at each terminal of the molecule.

Crosslinking diester compounds by produced by reaction of the above-mentioned unsaturated initiator with the above-mentioned polyalkylene glycol as an object of the present invention may be mentioned as the above-mentioned diesters, for example. Di(meth)alkyl ethers, vinyl ether-containing compounds and the like may be mentioned as the above-mentioned di(alkyl) ethers, for example. Ether compounds forming a dimer by bonding of two molecules of the above-mentioned polyalkylene glycols, ether compounds byproduced by bonding of two molecules of the above-mentioned active hydrogen compounds, and ether compounds generated by reaction of the above-mentioned polyalkylene glycols with the above-mentioned active hydrogen compounds also need to be reduced.

The above-mentioned content of the polymerizable dimer is preferably measured by high speed liquid chromatography method. For example, the content of the polymerizable dimer is preferably measured using the following measurement device and under the following conditions.
Device: product of TOSOH Corp., CCP & 8020 series, product of Shiseido Co., Ltd., Capsclpak C18
UG120 column (φ 4.6 mm×150 mm)
Conditions: Column temperature 40° C.
Eluent: H₂O/CH3CN=65/35 (ratio by volume)
Flow rate: 1 ml/min

In the polyalkylene glycols of the present invention, the above-mentioned polymerizable dimer content is preferably 0.001 to 10% by weight, and more preferably 0.001 to 3.5% by weight, and still more preferably 0.01 to 1.0% by weight, and particularly preferably 0.01 to 0.7% by weight.

If the polymerizable dimer content is reduced as mentioned above, the molecular weight distribution can be narrow, and gelling of the polymer can be prevented. Therefore, performances of a cement dispersant can be improved. The polyalkylene glycols of the present invention have high transparency and sufficient dispersibility because the above-mentioned polymerizable dimer is sufficiently reduced. The above-mentioned polyalkylene glycols can be produced by the production method of the present invention.

Accordingly, the preferable embodiments of the present invention include an embodiment in which the polyalkylene glycol of the present invention is a polyalkylene glycol produced by the production method of the present invention.

The preferable embodiments of the present invention include an embodiment in which the polymerizable polyalkylene glycol is produced by reacting an alkylene oxide with an unsaturated initiator, and 2% by weight of a polymerizable polyalkylene glycol in which an addition number of the alkylene oxide molecules to 1 mol of the unsaturated initiator is 15 to 20 mol is contained in 100% by weight of the polymerizable polyalkylene glycol.

If the polymerizable polyalkylene glycol contains 2% by weight or more of a polymerizable polyalkylene glycol in which a molar number of addition of the alkylene oxide to 1 mol of the unsaturated initiator is 15 to 20 mol (15 mol or more and 20 mol or less), a dispersant exhibiting higher dispersibility can be obtained. The polymerizable polyalkylene glycols of the present invention not only have a large molecular weight but also have a sufficiently reduced polymerizable dimer content, as mentioned above. And the glycols are not easily gelled and exhibit high dispersibility because containing 2% by weight or more of a polymerizable polyalkylene glycol in which a molar number of addition of the alkylene oxide to 1 mol of the unsaturated initiator is 15 to 20 mol. The function as a dispersant is believe to be largely a result of effects of the above-mentioned polymerizable polyalkylene glycol in which a molar number of addition of the alkylene oxide to 1 mol of the unsaturated initiator is 15 to 20 mol.

It is preferable that the polymerizable polyalkylene glycol contains 2% by weight or more of a polymerizable polyalkylene glycol in which a molar number of addition of the alkylene oxide to 1 mol of the unsaturated initiator is 15 to 20 mol. More preferably, the content of such a polyalkylene glycol is more preferably 5% by weight or more, and still more preferably 10% by weight or more, and particularly preferably 25% by weight or more, and most preferably 50% by weight or more.

Other additives may be added to the polyalkylene glycol of the present invention unless the functional effects of the present invention are sacrificed. Examples of polymerization inhibitors include methoquinone (hydroquinone monomethyl ether), BHT (2,6-di-tert-butylated hydroxytoluene), BHA (di-tert-butylhydroxyanisol), α-tocopherol, β-tocopherol, γ-tocopherol, and hydroquinone. These may be used in combination. Methoquinone (hydroquinone monomethyl ether) and BHT (2,6-di-tert-butylated hydroxytoluene) are preferable.

The polyalkylene glycols of the present invention hardly contain diols such as polyethylene glycol, polymerizable dimers such as diesters, metals, water, coloring-causing, substances. Therefore, such polyalkylene glycols of the present invention are used as a raw material to produce good resins. More specifically, the polyalkylene glycols of the present invention can be used as a raw material of transparent resins, plastic optical materials, urethane resins, UV-curable resins, resist materials, materials for electrochemical devices (secondary battery, capacitor, and the like), primers for woody materials, raw materials for curable resin compositions (coating material and the like), and adhesives for dentistry technology. Thus, the polyalkylene glycols of the present invention are polymerized to serve as a raw material of resins which can be used in various applications. That is, the present invention also includes a polyalkylene glycol polymer produced by polymerizing a monomer component comprising the polymerizable polyalkylene glycol of thee present invention.

The present invention also includes a use method of the polyalkylene glycol polymer, wherein the polyalkylene glycol polymer is used in an application of a dispersant, a cement admixture, a urethane resin, or an optical material.

That is, the preferable embodiments of the present invention include a dispersant, a cement admixture, a urethane resin, or a resin for optical materials each comprising the above-mentioned polyalkylene glycol polymer.

The production method of polyalkylene glycols of the present invention has the above-mentioned configuration and provide polyalkylene glycols which can be used as various industrial chemical raw materials and polyalkylene glycols having an unsaturated bond which are useful as monomers and the like forming polymers used in various application. The method is also a method for producing a polyalkylene glycol in which an alkylene oxide is effectively added to an unsaturated initiator and a content of byproducts is suppressed.

The polyalkylene glycols of the present invention have the above-mentioned configuration. And such glycols hardly contain impurities, and have good hue. Therefore, such glycols are preferable as a production raw material which can be used in various applications such as various polymer materials, concrete admixtures, cohesive agents, adhesive agents, coating materials, cosmetics additives, and various dispersants such as inorganic dispersants.

### BEST MODE FOR CARRYING OUT THE INVENTION

The present invention is described in more detail below with reference to Examples, but the present invention is not limited to the following Examples. The term “%” represents “% by weight” unless otherwise specified.

“Calculation Method of Conversion Rate”

A polyalkylene glycol was produced (after the reaction) and then hydroxyethyl methacrylate was quantified by gas chromatography to calculate a conversion rate at a ratio by weight to charged hydroxyethyl methacrylate.
Device: product of Simadzu Corp., GC-15A, product of J&W Capillary Columns & Accessories, capillary column DB-1 (0.53 mm φ×30 m)
Conditions: maintained at 40 ° C. for 5minutes, raised at 10° C./min., maintained at 300° C. for 5 minutes.
   “Moisture Content Measurement”

Measurement was performed using the following device.
Device: product of KYOTO ELECTRONICS MANUFACTURING (KEM) CO., LTD., MK-510
Standard sample: product of Mitsubishi Chemical, Karl Fisher SS
   “Hue Measurement”

About 30 cc of the sample was taken and subjected to measurement based on Hazen unit color number (platinum-cobalt scale) of color test method of chemicals according to JIS K 0071-1 (1998).

“Polymerizable Dimer Content Measurement”

Measurement was performed using the following measurement device and under the following measurement conditions.
Device: Product of TOSOH Corp., CCP & 8020 series, product of Shiseido Co., Ltd., Capsclpak C18
UG120 column (φ 4.6 mm×150 mm)
Conditions: The column temperature was 40° C. H₂/CH₃CN=65/35 (ratio by volume) was used as an eluent. The flow rate was 1 ml/min.
   “Metal Content Measurement”

The metal content was measured by ICP method.
Device: product of Rigaku Corp., CIROS-120

### EXAMPLE 1

(HEMA-3EO adduct reaction)

Into a SUS-made autoclave (1000 cc) were charged hydroxyethyl methacrylate (HEMA) 329.7 g (2.53 mol), methoquinone 0.1648 g, and silica alumina (product of NIKKI CHEMICAL CO., LTD., N633HN, calcinated at 350° C. for 4 hours) 33.2 g at room temperature. The container was sealed and substituted with nitrogen. The inner temperature of the reactor was heated to 70° C., and then a total of 449.1 g (10.2 mol) of ethylene oxide was charged over 4.7 hours. Then, the mixture was matured for 1.3 hours and then cooled and residual ethylene oxides were purged. The reaction solution obtained after pressure releasing was filtered through a 4μ filter paper to obtain transparent liquid with no color.

(Low-Boiling Cut)

This liquid was charged into a 1 L-glass container and distilled under 20 mm Hg of reduced pressure at a bath temperature of 80° C. to obtain liquid 570.7 g at the bottom. Thereby, a polyalkylene glycol (1) was obtained.

“Analysis Results”

The obtained polyalkylene glycol (1) was analyzed. The moisture content was measured by Karl Fischer method; the hue was measured by Hazen method; and the polymerizable dimer content and the polyethylene glycol content were measured by high performance liquid chromatography method. The above-mentioned measurement devices and the like were used.

The moisture content was 0.07% by weight, and the hue APHA was not Hazen of not less than 10, but less than 20. The polymerizable dimer content was 0.14% by weight, and no metal was detected. The effective component content was 88.0% by weight. The “effective component” used herein means polyethylene glycol compounds having a methacrylate structure at the terminal.

An aqueous solution (5 wt %) of the obtained polyalkylene glycol (1) was measured for pH at 25° C. The pH was 6.6.

### EXAMPLE 2

(Preparation of Silica-Supported Al Catalyst for HEMA-9EO Synthesis)

Aluminum nitrate 9-hydrate (product of Wako Pure Chemical Industries, Ltd.) 37.1 g was dissolved in water 1 L, and then silica (product of FUJI SILYSIA CHEMICAL LTD., CARiACT•Q-6) 200.0 g was added at room temperature and the mixture was stirred. Then, under stirring, water was distilled off under reduced pressure at 70° C. White powders obtained were dried at 120° C. for 8 hours under dry air flow, and then calcined for 4 hours at 550° C. Thereby, a silica-supported Al catalyst was produced.

(HEMA-9EO Addition Reaction)

Into a 10 mL-autoclave were charged hydroxyethyl methacrylate (HEMA, 1494.7 g, 11.48 mol), p-methoxyphenol (1.50 g, 121 mmol), the above-mentioned catalyst (the silica-supported Al catalyst, 150.65 g) at room temperature. The oxygen concentration of the gas phase was adjusted to 2% and the gage pressure was set to 1.0 kg·cm⁻². Under stirring, ethylene oxide (EO, 4572.2 g, 103.79 mol) was added at 70° C. to perform reaction for 19.2 hours. After the reaction, the reactant 5963.8 g was obtained. This liquid was analyzed by gas chromatography, which showed that the conversion rate of the raw material HEMA was 95.9%. The analysis of liquid chromatography showed that the polymerizable dimer content was 0.29 wt %. The analysis of Karl Fischer (KF) method showed that the moisture content was 0.20 wt %.

(Low-Boiling Cut)

Into a 1L-flask were charged the above-mentioned reactant 709.1 g and water 35.5 g. Then, this reaction mixtures were distilled at a pressure of 20 mm Hg and a bottom temperature of 25 to 80° C. Thereby, a polyethylene glycol (2) was produced.

(Analysis Results)

The polyethylene glycol (2) obtained through the removal of the low boiling contents was analyzed. As a result of the analysis, recovered was purified solution 596.6 q with an effective component of 85.3 wt %, a polymerizable dimer content of 0.66 wt %, and a moisture content of 0.16wt %. The “component” used herein means a polyethylene glycol compound having a methacrylate structure at the terminal. The hue APHA was not less than 10, but less than 20. No metal was detected.

### REFERENCE EXAMPLE 1

Into an autoclave were charged hydroxyethyl methacrylate 20.0 g, hydroquinone 0.026 g, tin chloride (SnC14, product of Wako Pure Chemical Industries, Ltd.) 0.20 g at room temperature. The gas phase was substituted with nitrogen gas, and the gage pressure was set to 0.15 MPa. Under stirring, ethylene oxide (EO) 20.4 g was injected over 2 hours at 50° C., and then the mixture was matured for 1.5 hours. Thereby, a polyethylene glycol (3) was obtained.

The obtained polyalkylene glycol (3) was analyzed, which showed that the polymerization dimer content was 3.6% by weight.

The metal amount could be calculated from the charged amount of Sn. The metal amount was 2243 ppm. The hue APHA was 50.

### COMPARATIVE EXAMPLE 1

Into an autoclave were charged hydroxyethyl methacrylate 95.4 g, benzoquinone 0.42 g, and double metal cyanide (DMC) catalyst 0.15 g at room temperature. Then, the gas phase was substituted with nitrogen gas and then the inside of the system was depressurized. Under stirring, ethylene oxide (EO) 49.1 g was charged over 6.2 hours at 100° C. while the inner pressure was kept to 0.1 MPa or less. However, the reaction did not proceed, and therefore feeding of the EO was interrupted. The DMC catalyst was prepared based on Examples in Japanese Kokai Publication No. Hei-08-0104741.

### EXAMPLE 3

(HEMA-9EO)

Methacrylic acid (MAA) 3.48 g and the polyalkylene glycol (2) obtained in Example 2 (HEMA-9EO: hydroxyethyl methacrylate-ethylene oxide adduct, the average molar number of addition of the ethylene oxide (EO) was 9) 12.30 g, and a 30 wt % aqueous solution of sodium hydroxide 0.28 g were added to water to prepare a total of 64 g of a monomer solution.

Ammonium persulfate (APS) 0.1551 g was added to water to prepare a total of 8 g of an initiator solution. 3-mercaptophenyl propionic acid (MPA) 0.3607 g was added to water to prepare a total of 8 g of a chain transfer solution.

The monomer solution was charged into a 100 mL-glass reactor in 35 mm diameter, and heated to 70° C. under stirring with a magnetic stirrer. Then, the initiator solution and the chain transfer solution were simultaneously added into the reactor to allow polymerization reaction to proceed. The temperature was kept to 70° C. for 2 hours to complete the polymerization reaction.

### EXAMPLE 4

(HEMA-9EO)

Polymerization was performed in the same manner as in Example 3, except that the amount of the chain transfer agent (MPA) was 0.1202 g.

### COMPARATIVE EXAMPLE 2

Polymerization was performed in the same manner as in Example 3, except that the polyalkylene glycol (3) obtained in Reference Example 1 was used instead of the polyalkylene glycol (2). The Mw became extremely large as shown in Table 1, because the polymerizable dimer content was large.

### COMPARATIVE EXAMPLE 3

Polymerization was performed in the same manner as in Comparative Example 2, except that the amount of the chain transfer agent was increased. As a result, a copolymer having almost the same Mw as in Example 3 was obtained.

### COMPARATIVE EXAMPLE 4

Polymerization was performed in the same manner as in Comparative Example 2, except that the amount of the chain transfer agent was decreased. As a result, the solution was gelled during the polymerization. Table 1 shows these results.

TABLE 1

| | MPA/g | Mw | Mn |
|---|---|---|---|
| Example 3 | 0.3607 | 58000 | 10300 |
| Example 4 | 0.1202 | 175000 | 78000 |
| Comparative | 0.3607 | 153000 | 54000 |
| Example 2 | | | |
| Comparative | 1.0821 | 56000 | 85000 |
| Example 3 | | | |

| | | |
|---|---|---|
| Comparative | 0.1202 | Incapable measurement |
| Example 4 | | due to gelling |

Table 1 is mentioned below. In Table 1, MPA represents the above-mentioned chain transfer agent. The value described in Table 1 is an amount (g) used at the polymerization. Mw is a weight average molecular weight. Mn is a number average molecular weight.

### EXAMPLE 5

Mortar Test Results

“Mortar Formulation”

Mortar formulation was C/S/W=550/1350/220 (g).

C, S, and W in the above formula mean those mentioned below, respectively.
C: Originally portland cement (product of TAIHEIYO CEMENT CORP.)
S: ISO standard sand
W: Polymer and ion exchange aqueous solution of antifoaming agent
   “Mortar Experimental Environment”

The experimental environment was 20° C.±1° C. and 60%±10% humidity.

“Mortar Kneading Procedure”

A 10% aqueous solution of the polymer obtained in Example 3, 10.18 g was added to 10 wt % relative to the polymer content of antifoaming agent MA-404 (product of Pozzolith Bussan Co., Ltd.) as it is. Thereto, ion exchange water was further added to prepare a mixture 220 g. The mixture was homogeneously dissolved sufficiently.

The mortar kneading procedures and the flow value measurement procedures were based on JIS R5201 (1997). N-50 mixer, product of HOBART (JAPAN) K.K., equipped with a stainless heater (stirrer blade) was used as a mixer.

“Mortar Air Amount Measurement Procedures”

The mortar about 200 mL was charged into 500 mL-Pyrex graduated cylinder and poked with a round bar in 8 mm diameter. Then, the container was vibrated to extract coarse air bubbles. Further, the mortar about 200 mL was added and air bubbles were similarly extracted, and then weighed. The air amount was calculated from the weight and the density of each of the materials.

“Molecular Weight of Copolymer and Molecular Weight Measurement Conditions”

Device: Waters Alliance (2605)
Analysis software: product of Waters, Empower professional+GPC option
Column: TSKgel, guard column (inner diameter 6.0×40 mm)+G4000 SWXL+G3000 SWXL+G2000 SWXL (each inner diameter 7.8×300 mm)
Detector: Differential refractometer (RI) detector (Waters 2414), multiwavelength visible ultraviolet (PDA) detector (Waters 2996)
Eluent: acetic acid was added to a mixture of acetonitrile/50 mM ion exchange aqueous solution of sodium acetate=40/60 (vol %) and thereby the pH was adjusted to 6.0.
Flow rate: 1.0 mL/min
Column and measurement temperature: 40° C.
Measurement time: 45 minutes
Injected amount of sample solution: 100 μL (0.5 wt % sample eluent solution)

GPC standard sample: 9 polyethylene glycols, products of TOSOH Corp., Mp=272500, 219300, 107000, 50000, 24000, 11840, 6450, 4250, 1470.
Calibration curve: prepared using Mp values of the above-mentioned polyethylene glycols and cubic polynomial.
Analysis method: In the obtained RI chromatogram, the polymer was detected and analyzed by connecting flatly stable parts to each other in the baselines immediately before and after the elution of the polymer. However, if the monomer peak and the polymer peak which overlap with each other were overlapped and measured, the molecular and the molecular weight distribution only in the polymer part was measured by perpendicularly dividing the most depressed part in the part where the monomer and the polymer overlap with each other, and thereby separating the polymer part from the monomer part. If a plurality of monomer peaks was detected, the monomer peak was divided from the polymer peak such that the monomer peak having the largest molecular weight was excluded. Oligomers not smaller than dimers were included in the polymer part.

### COMPARATIVE EXAMPLE 5

“Mortar Test”

Mortar test was performed using the comparative copolymer in Comparative Example 2.

### COMPARATIVE EXAMPLE 6

Mortar test was performed using the comparative copolymer in Comparative Example 3. Table 2 shows these results.

TABLE 2

| | | | | | Flow value | |
|---|---|---|---|---|---|---|
| | | Measurement | | Flow value | after 15 | |
| | Addition amount/ | time | Mortar | after 0 hit/ | hits/ | Air amount/ |
| | wt % to C | (minute) | temperature (° C.) | mm | mm | Vol % |
| Example 5 | 0.185 | 6 | 19.5 | 151 | 208 | 2.9 |
| Comparative | 0.185 | 6 | 19.5 | 125 | 163 | 3.0 |
| Example 5 | | | | | | |
| Comparative | 0.185 | 6 | 19.5 | 135 | 182 | 3.2 |
| Example 6 | | | | | | |

Table 2 is mentioned below.

In Table 2, “addition amount/wt % to C” means a ratio by weight (%) of the polymer relative to the above-mentioned originally Portland cement. The term “flow value after O hit” means an average value of the longest diameter and a diameter at 90° C. from the longest diameter of the mortar spreading over flow table when a flow cone was vertically lifted. The term “flow value after 15 hits” means an average value of a diameter and a diameter at 90° C. from the diameter of the mortar spreading after the flow table was moved up and down 15 times.

### EXAMPLE 6

A polyalkylene glycol (4) was prepared by adding methoquinone as a polymerization inhibitor to the polyalkylene glycol obtained in Example 1 such that the polyalkylene glycol (4) contains 750 ppm of the methoquinone. The obtained polyalkylene glycol (4) 5.0 g was charged into a glass test tube equipped with a thermometer. Thereinto, water containing 1.5% by weight of V-50 (product of Wako Pure Chemical Industries, Ltd., granulated product) 1.0 g was added as a polymerization initiator and nitrogen was bubbled thereinto. Then, polymerization was performed in an oil bath at 55° C. The temperature showed the highest value of 82.3° C. in 13 minutes, which showed that the polymerization had proceeded. The obtained polymer was a transparent solid with no color.

The copolymer of the present invention in Example 5 has a flow value larger than that in the comparative copolymers in. Comparative Examples 5 and 6. Such a result shows that the copolymer of the present invention is more excellent in dispersibility as a cement dispersant.

This Nonprovisional application claims priority under 35 U.S.C. § 119 on Patent Application No. 2005-252877 filed in Japan on Aug. 31, 2005, entitled “POLYALKYLENE GLYCOLS,”.

1. A polymerizable polyalkylene glycol satisfying the following characteristics (1) and/or (2):
(1) a polymerizable dimer content is 0.001 to 10% by weight; and
(2) a metal content is 50 ppm or less.

2. The polymerizable polyalkylene glycol according to claim 1,
wherein the polymerizable dimer content is 0.001 to 3.5% by weight.

3. The polymerizable polyalkylene glycol according to claim 1,
wherein the polymerizable polyalkylene glycol satisfies the characteristics (1) and (2), and the polymerizable dimer content is 0.001 to 3.5% by weight.

4. The polymerizable polyalkylene glycol according to claim 1,
wherein the polymerizable polyalkylene glycol has a hue (APHA value) of 1 to 40.

5. The polymerizable polyalkylene glycol according to claim 1,
wherein the polymerizable polyalkylene glycol is produced by reacting an alkylene oxide with an unsaturated initiator, and
2% by weight of a polymerizable polyalkylene glycol in which an addition number of the alkylene oxide molecules to 1 mol of the unsaturated initiator is 15 to 20 mol is contained in 100% by weight of the polymerizable polyalkylene glycol.

6. A method for producing a polyalkylene glycol by reacting an alkylene oxide with an unsaturated initiator,
wherein at least one solid acid catalyst selected from the group consisting of crystalline metal oxides and aluminum compounds having a 5-coordination structure of aluminum.

7. A polyalkylene glycol polymer produced by polymerizing a monomer component comprising the polymerizable polyalkylene glycol of claim 1.

8. A use method of the polyalkylene glycol polymer of claim 7, wherein the polyalkylene glycol polymer is used in an application of a dispersant, a cement admixture, an urethane resin, or an optical material.

9. The polymerizable polyalkylene glycol according to claim 2,
wherein the polymerizable polyalkylene glycol satisfies the characteristics (1) and (2), and the polymerizable dimer content is 0.001 to 3.5% by weight.

10. The polymerizable polyalkylene glycol according to claim 2,
wherein the polymerizable polyalkylene glycol has a hue (APHA value) of 1 to 40.

11. The polymerizable polyalkylene glycol according to claim 3,
wherein the polymerizable polyalkylene glycol has a hue (APHA value) of 1 to 40.

12. The polymerizable polyalkylene glycol according to claim 2,
wherein the polymerizable polyalkylene glycol is produced by reacting an alkylene oxide with an unsaturated initiator, and
2% by weight of a polymerizable polyalkylene glycol in which an addition number of the alkylene oxide molecules to 1 mol of the unsaturated initiator is 15 to 20 mol is contained in 100% by weight of the polymerizable polyalkylene glycol.

13. The polymerizable polyalkylene glycol according to claim 3,
wherein the polymerizable polyalkylene glycol is produced by reacting an alkylene oxide with an unsaturated initiator, and
2% by weight of a polymerizable polyalkylene glycol in which an addition number of the alkylene oxide molecules to 1 mol of the unsaturated initiator is 15 to 20 mol is contained in 100% by weight of the polymerizable polyalkylene glycol.

14. The polymerizable polyalkylene glycol according to claim 4,
wherein the polymerizable polyalkylene glycol is produced by reacting an alkylene oxide with an unsaturated initiator, and
2% by weight of a polymerizable polyalkylene glycol in which an addition number of the alkylene oxide molecules to 1 mol of the unsaturated initiator is 15 to 20 mol is contained in 100% by weight of the polymerizable polyalkylene glycol.

15. A polyalkylene glycol polymer produced by polymerizing a monomer component comprising the polymerizable polyalkylene glycol of claim 2.

16. A polyalkylene glycol polymer produced by polymerizing a monomer component comprising the polymerizable polyalkylene glycol of claim 3.

17. A polyalkylene glycol polymer produced by polymerizing a monomer component comprising the polymerizable polyalkylene glycol of claim 4.

18. A polyalkylene glycol polymer produced by polymerizing a monomer component comprising the polymerizable polyalkylene glycol of claim 5.

19. A use method of the polyalkylene glycol polymer of claim 15, wherein the polyalkylene glycol polymer is used in an application of a dispersant, a cement admixture, an urethane resin, or an optical material.

20. A use method of the polyalkylene glycol polymer of claim 16, wherein the polyalkylene glycol polymer is used in an application of a dispersant, a cement admixture, an urethane resin, or an optical material.
